(19) United States
(12) Patent Application Publication
(10) Pub. No.: US 2022/0395876 A1
(43) Pub. Date: Dec. 15, 2022

(54) APPARATUS AND METHOD FOR THERMAL PROCESSING OF MEDICAL WASTE

(71) Applicant: CONSOLIDATED BIOMEDICAL SOLUTIONS, INC., Delray Beach, FL (US)

(72) Inventors: Richard COHEN, Delray Beach, FL (US); Marcus Alex CASH, Delray Beach, FL (US); David FATER, Delray Beach, FL (US)

(21) Appl. No.: 17/775,442

(22) PCT Filed: Nov. 10, 2020
(86) PCT No.: PCT/US20/59793

§ 371 Date: May 9, 2022

Related U.S. Application Data
(60) Provisional application No. 62/933,796, filed on Nov. 11, 2019.

(51) Int. Cl. B09B 3/40; F23G 5/027; A61L 9/20

(52) U.S. Cl. CPC B09B 3/40; F23G 5/0276; A61L 9/20; F23G 2209/20

(57) ABSTRACT

Various implementations include an apparatus for thermal processing of medical waste. The apparatus includes a body, a conductive heater, and an air flow device. The body portion defines a chamber for receiving two or more containers of medical waste. The chamber is in fluid communication with a gas processing device for biological materials. The conductive heater is thermally coupled to the chamber to provide heat to the two or more containers. The air flow device is configured to direct discharge gases from the two or more containers to the gas processing device.

### BACKGROUND

Current solutions to disposal of medical waste include disposal of sharps and other medical waste in biohazard containers. Disposal companies can then collect these containers of sharps and medical waste to be autoclaved or incinerated off-site. However, this process can be costly and may not be practicable for remote locations.

Some current on-site medical waste disposal solutions include large devices capable of sterilizing a single, one-gallon standard sharps container of waste on-site. However, these machines can be too large to keep on a countertop and do not have the versatility to dispose of additional medical waste in the device during processing.

Thus, a need exists for a device that is small enough to keep in an office and allows for additional medical waste disposal during processing.

### SUMMARY

Various implementations include an apparatus for thermal processing of medical waste. The apparatus includes a body, a conductive heater, and an air flow device. The body portion defines a chamber for receiving two or more containers of medical waste. The chamber is in fluid communication with a gas processing device. The conductive heater is thermally coupled to the chamber to provide heat to the two or more containers. The air flow device directs discharge gases from the two or more containers to the gas processing device.

In some implementations, the gas processing device includes a filtration device for biological materials. In some implementations, the filtration device is a dual stage filter. In some implementations, the dual stage filter includes a first component having an antibacterial/antiviral device and a second component having an odor-trapping material.

In some implementations, the apparatus further includes a generator configured to combust fuel to create electricity for powering the two or more conductive heaters. In some implementations, the gas processing device includes a connecting pipe in fluid communication with the chamber and the generator. The air flow device directs at least a portion of the gases from the two or more containers to the generator and the generator combusts the gases as at least a portion of the fuel.

In some implementations, the chamber includes two or more compartments. Each of the two or more compartments is shaped to receive one of the two or more containers.

In some implementations, the body further includes a lid for sealingly covering the chamber. In some implementations, the body further includes two or more lids for sealingly covering the chamber. Each of the two or more lids is for sealingly covering one of the two or more compartments of the chamber.

In some implementations, the air flow device creates a negative pressure over the two or more containers to direct discharge gases from the two or more containers.

In some implementations, the conductive heater is configured to heat the chamber to a temperature of not less than 350° F. and not more than 385° F. In some implementations, the conductive heater includes two or more conductive heaters. Each of the two or more conductive heaters is thermally coupled to one of the two or more compartments. In some implementations, the apparatus further includes a staged-switching circuit configured to alternate providing power between each of the two or more conductive heaters.

In some implementations, each of the two or more containers has a volume of 5 gallons or more.

In some implementations, the apparatus further includes a UV-C light source for directing UV-C light into the chamber. Each of the two or more containers includes a UV-transparent material.

In some implementations, the chamber includes an antifriction coating.

In some implementations, the apparatus further includes a code scanner for reading scannable codes included on the two or more containers. In some implementations, the code scanner scans the scannable codes of the two or more containers when the two or more containers are disposed within the chamber.

In some implementations, the apparatus further includes a labeler for labeling the two or more containers with a readable label. In some implementations, the labeler labels the two or more containers when the two or more containers are disposed within the chamber.

Various other implementations include a process for heat-processing medical waste in an apparatus having a chamber. The process includes heating two or more containers of waste in the chamber using a conductive heater thermally coupled to the chamber, thereby rendering the waste biologically safe; directing discharge gases from the two or more containers in a predetermined direction; and processing the discharge gases with a gas processing device.

In some implementations, the gas processing device includes a filtration device for biological materials. In some implementations, the filtration device is a dual stage filter. In some implementations, the dual stage filter includes a first component having an antibacterial/antiviral device and a second component having an odor-trapping material.

In some implementations, heating the two or more containers of waste in the chamber further includes combusting fuel in a generator to create electricity for powering the two or more conductive heaters. In some implementations, the gas processing device includes a connecting pipe in fluid communication with the chamber and the generator. At least a portion of the discharge gases are directed from the two or more containers to the generator and the generator combusts the gases as at least a portion of the fuel.

In some implementations, the chamber includes two or more compartments. Each of the two or more compartments is shaped to receive one of the two or more containers.

In some implementations, directing discharge gases from the two or more containers creates a negative pressure over the two or more containers.

In some implementations, the waste includes an amount of thermoplastic material which melts upon the heating step. In some implementations, the process further includes the step of hardening the melted thermoplastic material to create a biologically sterile unitary mass in which sharp edges and points are at least partially encapsulated. In some implementations, each of the two or more containers of waste has an opening, and the unitary mass is larger than the opening of each of the two or more containers.

In some implementations, the conductive heater heats the two or more containers of waste to a temperature of not less than 350° F. and not more than 385° F.

In some implementations, the conductive heater includes two or more conductive heaters. Each of the two or more conductive heaters is thermally coupled to one of the two or more compartments. In some implementations, the process further includes a staged-switching circuit configured to alternate providing power between each of the two or more conductive heaters.

In some implementations, each of the two or more containers has a volume of 5 gallons or more.

In some implementations, the process further includes directing UV-C light created by a UV-C light source into the chamber. Each of the two or more containers includes a UV-transparent material.

In some implementations, the chamber comprises an antifriction coating.

In some implementations, the process further includes reading a scannable code included on each of the two or more containers using a code scanner. In some implementations, the code scanner scans the scannable codes of the two or more containers when the two or more containers are disposed within the chamber.

In some implementations, the process further includes labeling the two or more containers with a readable label using a labeler. In some implementations, the labeler labels the two or more containers when the two or more containers are disposed within the chamber.

Various other implementations include an apparatus for thermal processing of medical waste. The apparatus includes a body portion, a conductive heater, and an air flow device. The body portion defines a chamber for receiving a container of medical waste. The chamber is in fluid communication with a gas processing device. The container has a volume of 5 gallons or more. The conductive heater is thermally coupled to the chamber to provide heat to the container. The air flow device directs discharge gases from the container to the gas processing device.

In some implementations, the gas processing device includes a filtration device for biological materials. In some implementations, the filtration device is a dual stage filter. In some implementations, the dual stage filter includes a first component having an antibacterial/antiviral device and a second component having an odor-trapping material.

In some implementations, the apparatus further includes a generator configured to combust fuel to create electricity for powering the conductive heater. In some implementations, the gas processing device includes a connecting pipe in fluid communication with the chamber and the generator. The air flow device directs at least a portion of the gases from the container to the generator and the generator combusts the gases as at least a portion of the fuel.

In some implementations, the body further includes a lid for sealingly covering the chamber.

In some implementations, the air flow device creates a negative pressure over the container to direct discharge gases from the container.

In some implementations, the conductive heater is configured to heat the chamber to a temperature of not less than 350° F. and not more than 385° F.

In some implementations, the apparatus further includes a UV-C light source for directing UV-C light into the chamber. The container includes a UV-transparent material.

In some implementations, the chamber includes an antifriction coating.

In some implementations, the apparatus further includes a code scanner for reading a scannable code included on the container. In some implementations, the code scanner scans the scannable code of the container when the container is disposed within the chamber.

In some implementations, the apparatus further includes a labeler for labeling the container with a readable label. In some implementations, the labeler labels the container when the container is disposed within the chamber.

Various other implementations include a process for heat-processing medical waste in an apparatus having a chamber. The process includes heating a container of waste in the chamber using a conductive heater thermally coupled to the chamber, thereby rendering the waste biologically safe, wherein the container has a volume of 5 gallons or more; directing discharge gases from the container in a predetermined direction; and processing the discharge gases with a gas processing device.

In some implementations, the gas processing device includes a filtration device for biological materials. In some implementations, the filtration device is a dual stage filter. In some implementations, the dual stage filter includes a first component having an antibacterial/antiviral device and a second component having an odor-trapping material.

In some implementations, heating the container of waste in the chamber further includes combusting fuel in a generator to create electricity for powering the conductive heater. In some implementations, the gas processing device includes a connecting pipe in fluid communication with the chamber and the generator. At least a portion of the discharge gases are directed from the container to the generator and the generator combusts the gases as at least a portion of the fuel.

In some implementations, directing discharge gases from the container creates a negative pressure over the container.

In some implementations, the waste includes an amount of thermoplastic material which melts upon the heating step. In some implementations, the process further includes the step of hardening the melted thermoplastic material to create a biologically sterile unitary mass in which sharp edges and points are at least partially encapsulated. In some implementations, the container of waste has an opening, and the unitary mass is larger than the opening of each of the container.

In some implementations, the conductive heater heats the container of waste to a temperature of not less than 350° F. and not more than 385° F.

In some implementations, the process further includes directing UV-C light created by a UV-C light source into the chamber. The container includes a UV-transparent material.

In some implementations, the chamber includes an antifriction coating.

In some implementations, the process further includes reading a scannable code included on the container using a code scanner. In some implementations, the code scanner scans the scannable code of the container when the container is disposed within the chamber.

In some implementations, the process further includes labeling the container with a readable label using a labeler. In some implementations, the labeler labels the container when the container is disposed within the chamber.

### BRIEF DESCRIPTION OF DRAWINGS

Example features and implementations are disclosed in the accompanying drawings. However, the present disclosure is not limited to the precise arrangements and instrumentalities shown. Similar elements in different implementations are designated using the same reference numerals.

FIG. 1A is a side view of an apparatus for thermal processing of medical waste including two lids, according to one implementation.

FIG. 1B is a side view of an apparatus for thermal processing of medical waste including one lid, according to another implementation.

FIG. 2 is a side view of an apparatus for thermal processing of medical waste including a chamber for receiving a 5-gallon container, according to another implementation.

FIG. 3 is a side view of an apparatus for thermal processing of medical waste including a generator, a connecting pipe, a UV-C light source, and a code scanner, according to another implementation.

### DETAILED DESCRIPTION

The devices, systems, and methods disclosed herein provide for a way to heat medical waste to a temperature, and hold the waste at the temperature for a length of time, such that the waste is rendered sterile and non-biohazardous. The device is small enough to keep in an office, providing for an economic safe, simple, and secure approach for on-site biomedical waste management. Once the waste is rendered sterile and non-biohazardous, the one or more containers can be disposed of on site in normal, single-stream trash. The multiple-container layout of the devices disclosed herein increases the maximum processing rate, and the larger container size allows for larger processing batches.

Various implementations include an apparatus for thermal processing of medical waste. The apparatus includes a body, a conductive heater, and an air flow device. The body portion defines a chamber for receiving two or more containers of medical waste. The chamber is in fluid communication with a gas processing device. The conductive heater is thermally coupled to the chamber to provide heat to the two or more containers. The air flow device directs discharge gases from the two or more containers to the gas processing device.

Various other implementations include a process for heat-processing medical waste in an apparatus having a chamber. The process includes heating two or more containers of waste in the chamber using a conductive heater thermally coupled to the chamber, thereby rendering the waste biologically safe; directing discharge gases from the two or more containers in a predetermined direction; and processing the discharge gases with a gas processing device.

Various other implementations include an apparatus for thermal processing of medical waste. The apparatus includes a body portion, a conductive heater, and an air flow device. The body portion defines a chamber for receiving a container of medical waste. The chamber is in fluid communication with a gas processing device. The container has a volume of 5 gallons or more. The conductive heater is thermally coupled to the chamber to provide heat to the container. The air flow device directs discharge gases from the container to the gas processing device.

Various other implementations include a process for heat-processing medical waste in an apparatus having a chamber. The process includes heating a container of waste in the chamber using a conductive heater thermally coupled to the chamber, thereby rendering the waste biologically safe, wherein the container has a volume of 5 gallons or more; directing discharge gases from the container in a predetermined direction; and processing the discharge gases with a gas processing device.

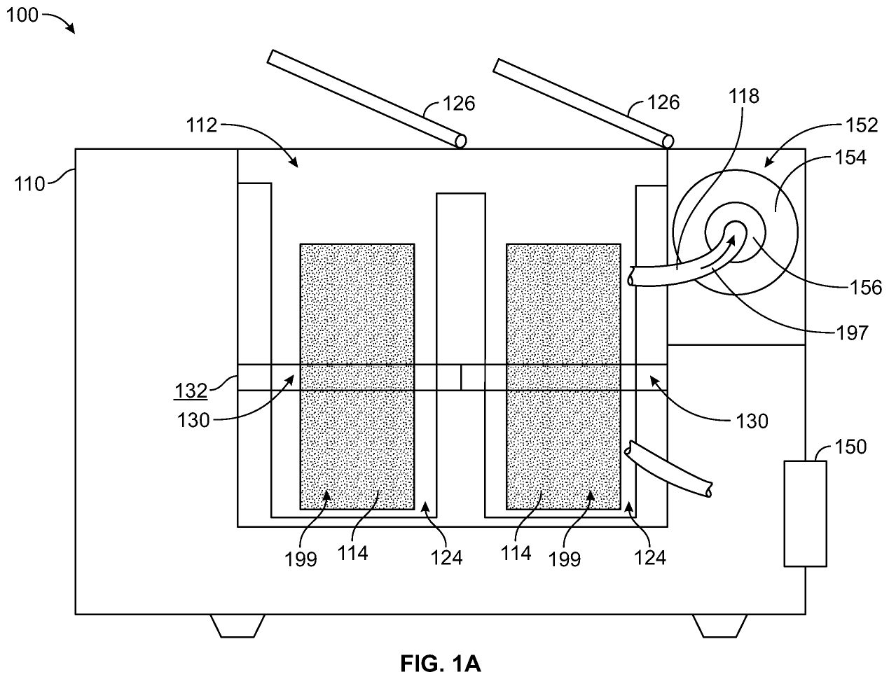

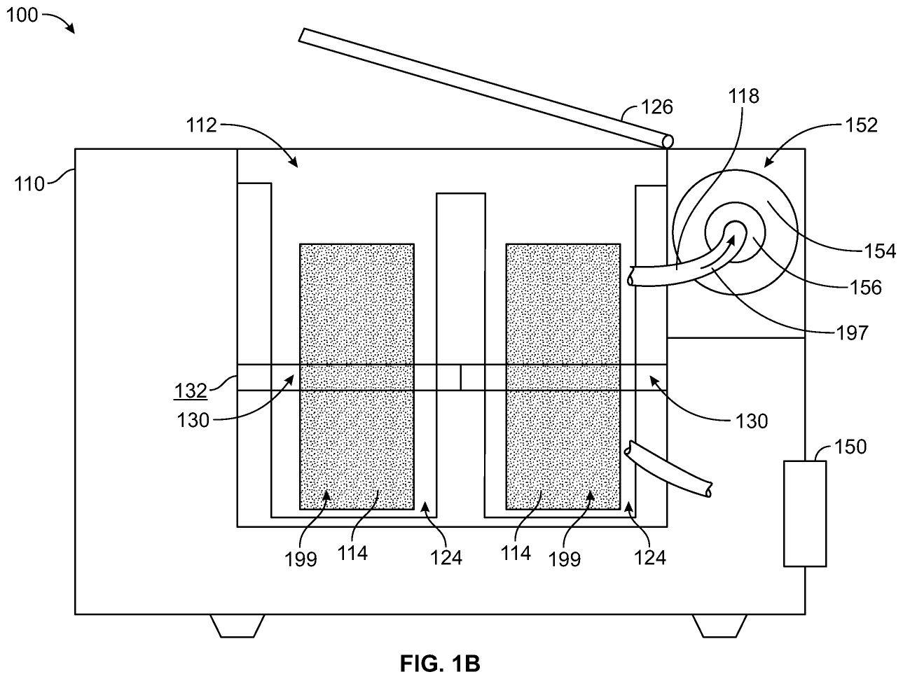

FIGS. 1A-3 show implementations of apparatuses 100 for thermal processing of medical waste 199. The apparatus 100 shown in FIGS. 1A-3 includes a body portion 110, a thermally conductive heater 130, and an air flow device 150. The thermally conductive 130 heater can be powered either by electric resistance or by fuel combustion (e.g., propane). The body portion 110 defines a chamber 112 for receiving two or more containers 114 of medical waste 199. The chamber 112 is in fluid communication with a filtration device 152 for biological materials. In some implementations, each of the two or more containers 114 has a volume of 5 gallons or more.

The chamber 112 of the apparatus 100 shown in FIGS. 1A-3 is in fluid communication with the filtration device 152 via a ducting system 118, but in other implementations, the chamber is in fluid communication with the filtration device via any other means known in the art.

The thermally conductive heater 130 shown in FIGS. 1A-3 is thermally coupled to the chamber 112 to provide heat to the two or more containers 114. The heat transferred to the chamber 112 is then transferred to the two or more containers 114 to maintain an elevated temperature range within each of the containers 114 for sterilizing and melting the medical waste 199. The conductive heater shown in FIGS. 1A-3 is an electric resistance heater in contact with a surface of the chamber to transfer heat through the chamber walls to the containers, but in other implementations, the conductive heater is any type of heater capable of conductively transferring heat from the heater to the chamber. In some implementations, the apparatus includes a thermally convective heater or a radiant heater to transfer heat to the chamber and/or the container. In some implementations, the conductive heater 130 is configured to heat the chamber 112 to a temperature of not less than 350° F. and not more than 385° F. The 350° F. minimum temperature ensures that the low-temperature thermoplastic waste melts, and the 385° F. maximum temperature ensures that the ancillary (device) plastics do not melt.

The air flow device 150 is configured to direct discharge gases 197 from the two or more containers 114 to the filtration device 152. The air flow device 150 shown in FIGS. 1A-3 is a fan that pulls air over the exhaust at the filter exit 152, creating a slight pressure drop in the chamber 112 (e.g., via the Venturi effect or Bernouli's principle). In some implementations, the air flow device 150 creates a negative pressure over the two or more containers 114 to direct discharge gases 197 towards the filter.

In some implementations, the filtration device 152 is a dual stage filter. In some implementations, the dual stage filter includes a first component 154 having an antibacterial/antiviral device and a second component 156 having an odor-trapping material. In the figures, the odor-trapping material is activated carbon, but in other implementations, the odor-trapping device can be any other material capable of removing odor from discharge gases. The antibacterial/antiviral device shown in the figures is an ultra-violet (“UV-C”) source for providing UV-C irradiation to the discharge gases, but in other implementations, the antibacterial/antiviral device is any device capable of sterilizing discharge gases.

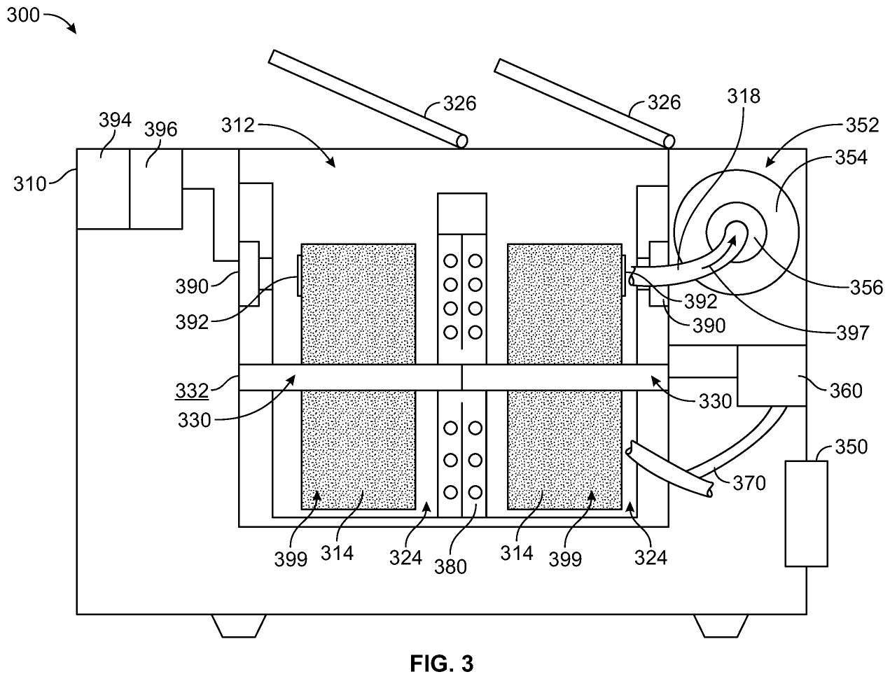

In the implementation shown in FIG. 3, the device includes a UV-C light source that directs UV-C light into the chamber. Each of the containers within the chamber is made of a UV-C transparent material such that the UV-C light emitted into the chamber penetrated through the containers to kill or disable bacteria or viruses in the waste within the containers.

In some implementations, the chamber 112 includes two or more compartments 124. Each of the two or more compartments 124 is shaped to receive one of the two or more containers 114. In the implementations shown in FIG. 1A, the body 110 includes two lids 126 for sealingly covering the chamber 112. Each of the two lids 126 is for sealingly covering one of the two or more compartments 124 of the chamber 112. FIG. 1B shows a device with one lid covering two compartments, but in other implementations, the device includes any number of lids for covering the compartments.

The implementations shown in FIGS. 1A and 1B include two conductive heaters, but in other implementations, the device includes two or more conductive heaters. Each of the two or more conductive heaters 130 is thermally coupled to one of the two or more compartments 124. In some implementations, the apparatus 100 further includes a staged-switching circuit 132 configured to alternate providing power between each of the two or more conductive heaters 130. The two or more compartments 124 shown in the figures are similarly shaped and sized to maximize heat transfer during heating of the containers 114, but in other implementations, the two or more compartments are various different shapes and sizes to accommodate different containers for different needs.

Various other implementations include a process for heat-processing medical waste in an apparatus 100 having a chamber 112. The process includes (1) heating two or more containers 114 of waste 199 in the chamber 112 using a conductive heater 130 thermally coupled to the chamber 112, thereby rendering the waste 199 biologically safe; (2) directing discharge gases 197 from the two or more containers 114 in a predetermined direction; and (3) filtering the discharge gases 197 with a filtration device 152.

In some implementations, the filtration device 152 is a dual stage filter. In some implementations, the dual stage filter includes a first component 154 having an antibacterial/antiviral device and a second component 156 having an odor-trapping material.

In some implementations, the chamber 112 includes two or more compartments 124. Each of the two or more compartments 124 is shaped to receive one of the two or more containers 114. In some implementations, the conductive heater 130 includes two or more conductive heaters 130. Each of the two or more conductive heaters 130 is thermally coupled to one of the two or more compartments 124. In some implementations, the conductive heater 130 further includes a staged-switching circuit 132 configured to alternate providing power between each of the two or more conductive heaters 130.

In some implementations, directing discharge gases 197 from the two or more containers 114 creates a negative pressure over the two or more containers 114.

In some implementations, the waste 199 includes an amount of thermoplastic material which melts upon the heating step. In some implementations, the process further includes hardening the melted thermoplastic material to create a biologically sterile unitary mass in which sharp edges and points are at least partially encapsulated. In some implementations, each of the two or more containers 114 of waste 199 has an opening, and the unitary mass is larger than the opening of each of the two or more containers 114.

In some implementations, the conductive heater 130 heats the two or more containers 114 of waste to a temperature of not less than 350° F. and not more than 385° F.

In some implementations, each of the two or more containers 114 has a volume of 5 gallons or more.

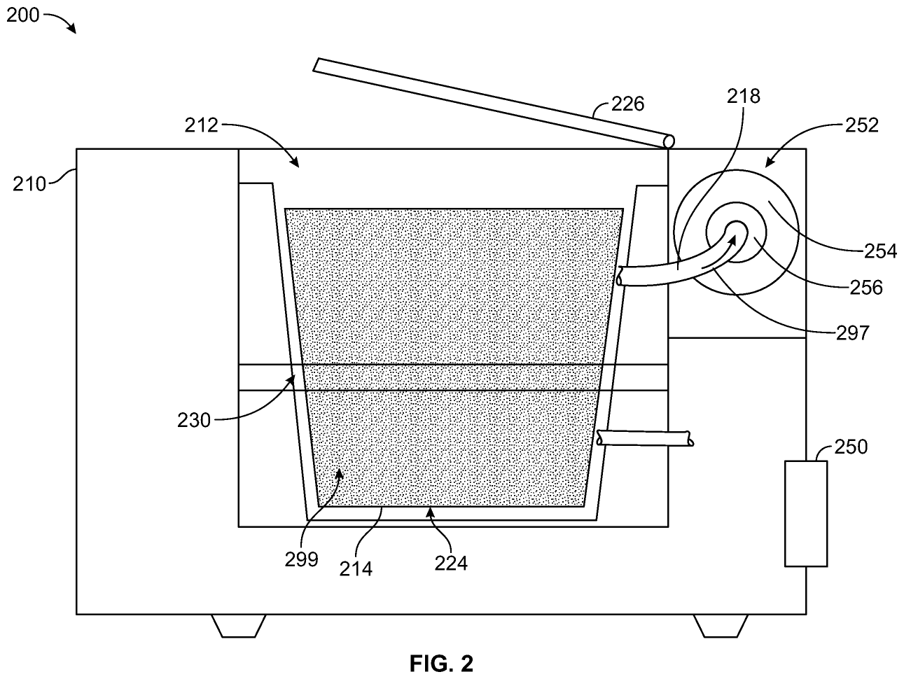

FIG. 2 shows an apparatus 200 for thermal processing of medical waste 299. The apparatus 200 includes a body portion 210, a conductive heater 230, and an air flow device 250. The body portion 210 defines a chamber 212 for receiving a container 214 of medical waste 299. The chamber 212 is in fluid communication with a filtration device 252 for biological materials 299. The container 214 has a volume of 5 gallons or more. The conductive heater 230 is thermally coupled to the chamber 212 to provide heat to the container 214. The air flow device 250 is configured to direct discharge gases 297 from the container 214 to the filtration device 252.

In some implementations, the filtration device 252 is a dual stage filter. In some implementations, the dual stage filter includes a first component 254 having an antibacterial/antiviral device and a second component 256 having an odor-trapping material.

In some implementations, the body 210 further includes a lid 226 for sealingly covering the chamber 212.

In some implementations, the air flow device 250 creates a negative pressure over the container 214 to direct discharge gases 297 towards the filter 252.

In some implementations, the conductive heater 230 is configured to heat the chamber 212 to a temperature of not less than 350° F. and not more than 385° F.

Various other implementations include a process for heat-processing medical waste 299 in an apparatus having a chamber 212. The process includes (1) heating a container 214 of waste 299 in the chamber 212 using a conductive heater 230 thermally coupled to the chamber 212, thereby rendering the waste 299 biologically safe, wherein the container 214 has a volume of 5 gallons or more; (2) directing discharge gases 297 from the container 214 in a predetermined direction; and (3) filtering the discharge gases 297 with a filtration device 252.

In some implementations, the filtration device 252 is a dual stage filter. In some implementations, the dual stage filter includes a first component 254 having an antibacterial/antiviral device and a second component 256 having an odor-trapping material.

In some implementations, directing discharge gases 297 from the container 214 creates a negative pressure over the container 214.

In some implementations, the waste 299 includes an amount of thermoplastic material which melts upon the heating step. In some implementations, the process further includes hardening the melted thermoplastic material to create a biologically sterile unitary mass in which sharp edges and points are at least partially encapsulated. In some implementations, the container 214 of waste 299 has an opening, and the unitary mass is larger than the opening of each of the container.

In some implementations, the conductive heater 230 heats the container 214 of waste 299 to a temperature of not less than 350° F. and not more than 385° F.

Although the devices and processes disclosed herein include containers having volumes of 5 gallons or more, in other implementations, the containers have any volume, such as less than 1 gallon, 1 gallon or more, 2 gallons or more, 3 gallons or more, 4 gallons or more, etc. The containers (and matching chamber) can be custom OEM-supplied or (depending on regulations) use locally-recycled common household or business/industry containers (e.g, paint, milk, detergent, 55-gallon chemical/petrol drums, etc.).

In some implementations, the chamber is shaped and sized to receive a container that is a standard shape and size as used throughout the small-medical office industry. In some implementations, the container includes a melting crucible rotating inlet vane which eliminates accidental “sticks” from sharps and forms additional protective thermoplastic coating on top of sharps' melted mass. If necessary, the container can be reused after any of the above processes when such consumables may not be readily available.

In some implementations, a thermal bimetallic cutoff is included on the chambers for additional protection in case of a thermocouple sensor failure. Line current sensor can also be included to allow the maximum current flow for rapid chamber heating without tripping a circuit breaker. As an additional safety feature, a particle sensor (operating like a smoke detector) can be included to monitor the discharge gases. In some implementations, thermal feedback can be provided via a thermocouple and/or an IR sensor (possible via fiber-optics) from each lateral side of each chamber. In implementations configured for two or more containers, proximity sensors can be included on each chamber, which can be used in conjunction with multiple heating coils to focus heat if all chambers are not used simultaneously. In some implementations, a use timer can be included to alert the user to change the charcoal filter and/or UV bulbs.

In some implementations, the conductive heater includes thermoelectric tiles lining the chamber periphery.

In some implementations, the apparatus includes an embedded arc-fault circuit breaker and coordinated higher capacity fast-blow fuse, which provide for maximum electrical protection. In some implementations, a thermal breaker (bimetallic strip) is included between chambers to protect against the malfunctioning of controls.

In some implementations, non-contact (proximity/magnetic/tilt) or contact-buffer lockout switches are included to reduce the susceptibility of mechanical wear or failure. In some implementations, the apparatus includes redundant (ball-bearing) exhaust and intake fans to reduce a means of mechanical failure. The apparatus can include a rubberized insert housing for condensate collection.

Current solutions to on-site disposal of medical waste include a device capable of sterilizing only a single, one-gallon standard sharps container of waste. U.S. Pat. No. 5,972,291 filed on Jan. 30, 1997, and titled “Method and Apparatus for Disposal of Infectious and Medical Waste” discloses this single, one-gallon container device, the contents of which are incorporated herein by reference.

However, an apparatus for thermal processing of medical waste that includes a chamber for receiving two or more containers of medical waste provides for distinct advantages over the prior art. The capability of heating two or more containers, either simultaneously or individually, allows for more waste disposal and greater flexibility. Furthermore, heating two or more containers requires more power draw. For many standard 110-volt circuits, simultaneously heating two or more containers would draw too many amps. The staged-switching circuit disclosed herein allows for simultaneous heating of two or more containers by alternating power delivered to each of the containers. In this way, the apparatus as a whole does not draw more amps than the circuit to which the apparatus is connected can supply.

An apparatus for thermal processing of medical waste that includes a chamber for receiving a 5-gallon or more container of medical waste also provides for distinct advantages over the prior art. Standard sharps containers have a 1-gallon volume and a specific shape. The standard sharps containers are a specific size and shape for various reasons, such as for convention for container accessories, because many medical facilities do not need larger containers in individual rooms, to meet counter space restrictions, etc. The standard 1-gallon containers also increase the ease of collection of the containers of third-party medical waste disposers. However, a larger container, such as the 5-gallon or more containers disclosed herein, are better suited for situations when a medical facility is disposing of the entirety of their sharps or other waste themselves.

FIG. 3 shows an apparatus 300 similar to the apparatus 100 shown in FIG. 1A, but the apparatus 300 shown in FIG. 3 also includes a generator 360, a connecting pipe 370, a UV-C light source 380, and a code scanner 390.

The generator 360 is included in the body portion 310 of the apparatus 300 and is configured to combust fuel to create electricity for powering the two or more conductive heaters 330. The generator 360 shown in FIG. 3 is configured to use propane and discharge gases as fuel, but in other implementations, the generator is configured to use gasoline, diesel, natural gas, or any other form of fuel known to be used in generators.

The connecting pipe 370 and the filtration device 352 are components of a gas processing device. However, in other implementations, the apparatus may include only one of a connecting pipe or a filtration device. The connecting device 370 is in fluid communication with the chamber 312 and the intake of the generator 360. The air flow device 350 directs at least a portion of the discharge gases 397 from the two containers 314 to the generator 360, and the generator 360 uses the discharge gases 397 as part of the fuel, combusting the gases 397 along with the propane. In some implementations, the combustion within the generator creates a negative pressure or vacuum that causes the flow of the discharge gases from the chamber to the generator.

The UV-C light source 380 is oriented within the body portion 310 of the apparatus 300 such that the UV-C light source 380 directs UV-C light through a UV-C transparent portion of the chamber wall and into the chamber 312. Each of the containers 314 is made of a UV-transparent material such that the UV-C light emitted from the UV-C light source 380 can penetrate into the waste 399. The UV-C light kills or disables the microbes within the waste 399. The UV-C light can be employed either as a pre-processing stage of the waste 399 prior to the heating of the waste 399 or in addition to the heating process. Using the UV-C light as a pre-processing of the waste 399 is particularly useful in larger containers 314, in circumstances where less waste is being generated, or in any other situation where there may be long periods between processing. The containers 314 shown in FIG. 3 are made of glass, but in other implementations, the UV-C transparent material could be plastic or any other material that allows UV-C light to penetrate inside of the containers to kill or disable microbes.

The code scanner 390 is included in the body portion 310 and is oriented to scan a code 392 on a container 314 when the container 314 is disposed within the chamber 312. The code 392 can include a tracking number or scannable code (e.g., a bar code or a QR code) on each container 314. In some implementations, the code scanner can be external to the chamber. The scannable code 392 can be embossed or engraved (e.g., by laser) in the container 314 for longevity and durability.

The apparatus 300 also includes a controller 394 and memory 396. When waste 399 is processed in a container 314 with a scannable code 392, information about the waste 399 contained in the container 314 can be entered into the apparatus with a keypad and that information is then associated with the scannable code 392. This information can then be stored either locally on the device's memory 396 or can be uploaded to a remote memory (e.g., cloud storage). The scannable codes 392 on each container 314 can also be used to track the number of unused containers 314 remaining and, in implementations that include the ability to upload scannable code data to a remote memory, this information can be used for automatic order placement for new containers 314 (e.g. a subscription service). The scannable code 392 facilitates both long-term regulatory tracking of the waste stream, as well as streamlining automatic ordering of replacement containers, as the machine logs and reports container usage post-processing.

In some implementations, information about the waste contained in the container can be entered into the device, and a printer can print a label containing the information that can be applied to the container.

In some implementations, the apparatus includes a combustion chamber that is used to directly heat the chambers by the combustion of fuel. In some implementations, a generator is included, as in FIG. 3, but the generator is a lower power rating generator. The generator only provides enough electricity to power the electronics of the apparatus, but the hot exhaust gases are ducted to the chamber to directly heat the chamber.

In some implementations, the chamber of the apparatus includes an antifriction coating, such as Teflon, and the chamber is a sealed or impervious (i.e., no seams) chamber that prevents the leakage of liquid and/or gas. In these implementations, the waste can be disposed directly into the chamber with a non-standard container or without a container. Once the waste has been processed and has melted into a solitary mass, the antifriction surface allows the waste to be easily removed from the chamber.

A number of implementations have been described. Nevertheless, it will be understood that various modifications may be made without departing from the spirit and scope of the claims. Accordingly, other implementations are within the scope of the following claims.

Certain terminology is used herein for convenience only and is not to be taken as a limitation on the present claims. In the drawings, the same reference numbers are employed for designating the same elements throughout the several figures. A number of examples are provided, nevertheless, it will be understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

1. An apparatus for thermal processing of medical waste comprising:
a body portion defining a chamber for receiving two or more containers of medical waste, the chamber being in fluid communication with a gas processing device;
a conductive heater thermally coupled to the chamber to provide heat to the two or more containers; and
an air flow device to direct discharge gases from the two or more containers to the gas processing device.

2. The apparatus of claim 1, wherein the gas processing device comprises a filtration device for biological materials.

3. The apparatus of claim 2, wherein the filtration device is a dual stage filter.

4. The apparatus of claim 3, wherein the dual stage filter comprises a first component having an antibacterial/antiviral device and a second component having an odor-trapping material.

5. The apparatus of claim 1, further including a generator configured to combust fuel to create electricity for powering the two or more conductive heaters.

6. The apparatus of claim 5, wherein the gas processing device comprises a connecting pipe in fluid communication with the chamber and the generator, wherein the air flow device directs at least a portion of the gases from the two or more containers to the generator and the generator combusts the gases as at least a portion of the fuel.

7. The apparatus of claim 1, wherein the chamber includes two or more compartments, each of the two or more compartments being shaped to receive one of the two or more containers.

8. The apparatus of claim 1, wherein the body further includes a lid for sealingly covering the chamber.

9. The apparatus of claim 7, wherein the body further includes two or more lids for sealingly covering the chamber, each of the two or more lids for sealingly covering one of the two or more compartments of the chamber.

10. The apparatus of claim 1, wherein the air flow device creates a negative pressure over the two or more containers to direct discharge gases from the two or more containers.

11. The apparatus of claim 1, wherein the conductive heater is configured to heat the chamber to a temperature of not less than 350° F. and not more than 385° F.

12. The apparatus of claim 7, wherein the conductive heater comprises two or more conductive heaters, each of the two or more conductive heaters being thermally coupled to one of the two or more compartments.

13. The apparatus of claim 12, further comprising a staged-switching circuit configured to alternate providing power between each of the two or more conductive heaters.

14. The apparatus of claim 1, wherein each of the two or more containers has a volume of 5 gallons or more.

15. The apparatus of claim 1, further comprising a UV-C light source for directing UV-C light into the chamber, wherein each of the two or more containers comprises a UV-transparent material.

16. The apparatus of claim 1, wherein the chamber comprises an antifriction coating.

17. The apparatus of claim 1, further comprising a code scanner for reading scannable codes included on the two or more containers.

18. The apparatus of claim 17, wherein the code scanner scans the scannable codes of the two or more containers when the two or more containers are disposed within the chamber.

19. The apparatus of claim 1, further comprising a labeler for labeling the two or more containers with a readable label.

20. The apparatus of claim 19, wherein the labeler labels the two or more containers when the two or more containers are disposed within the chamber.

21. A process for heat-processing medical waste in an apparatus having a chamber, comprising the steps of:
heating two or more containers of waste in the chamber using a conductive heater thermally coupled to the chamber, thereby rendering the waste biologically safe;
directing discharge gases from the two or more containers in a predetermined direction; and
processing the discharge gases with a gas processing device.

22.-73. (canceled)
